(19) United States
(12) Patent Application Publication
(10) Pub. No.: US 2011/0270203 A1
(43) Pub. Date: Nov. 3, 2011

(54) External voiding apparatus and system

(75) Inventors: Isabella K. Sharpe, Jacksonville, FL (US); Eric P. Stord, Melbourne, FL (US)

(73) Assignee: SHARPE IDEAS, INC.

(21) Appl. No.: 12/799,632

(22) Filed: Apr. 28, 2010

(51) Int. Cl. A61F 5/451

(52) U.S. Cl. 604/326; 604/347

(57) ABSTRACT

An external voiding apparatus includes a triangular cup, an outlet at a vertex of the cup, a throat extending outwardly from the outlet of the cup, a check valve assembly, and a valve seat. The cup has an open top and gradually sloping walls that converge at the outlet adjacent a lower vertex of the cup. The valve assembly is disposed between the throat and the valve seat. A tube and a fluid coupling assembly connect (i.e., fluidly couple) the valve seat to the tube. The check valve may comprise a valve body with an aperture covered downstream by an easily deflectable membrane, or a duckbill valve. The fluid coupling assembly includes a funnel coupling from the reservoir to a tube. A fluid collection container (e.g. a Foley bag) may be fluidly coupled to the tube. The cup may include a phosphorescent polymer to provide a glow in the dark effect, a handle attached to the cup and a removable cover. A wide compressible resilient gasket is provided on the top edge of the cup, which may be anatomically contoured. A support member suitable for holding the aforesaid apparatus in convenient reach of the user and/or associated care givers completes the system of the invention.

### FIELD OF THE INVENTION

This invention relates generally to urinals, and more particularly, to an external voiding apparatus and system suitable for capturing urine from female and male users.

### BACKGROUND

Various devices have been developed for collecting urine from bedridden female and male patients. Such devices typically comprise a container with an inlet configured to channel the flow of urine to a container. By way of example, US Publication No. 2006/0155214 discloses a urine collection device with a funnel connected to a container by a hose. U.S. Pat. Nos. 3,927,426 and 4,270,231 disclose urinals with hinged closures. U.S. Pat. No. 4,568,339 discloses an hourglass shaped funnel with a deflectable rim and an optional gasket. U.S. Pat. No. 4,764,991 discloses a flexible funnel connected to a container with a hose. U.S. Pat. No. 4,771,484 discloses a rigid funnel surrounded by a flexible funnel sleeve connected to a hose. U.S. Pat. No. 4,936,838 discloses an hourglass shaped funnel with an annular rim that contacts the wearer. A longitudinal ridge extends from the funnel between the buttocks of the user. U.S. Pat. No. 5,295,983 discloses a urinary collector worn under clothing and undergarments to receive urinary discharges and snugly fits directly around a woman's vulva and urethral meatus. The collector is formed of soft, flexible water-proof material and contains absorbent hydrophobic material and a discharge outlet. U.S. Pat. No. 5,592,699 discloses a mouth piece for attachment to the mouth of a urinal. The mouth piece includes a rim for sealing contact with a user. A liquid barrier or bridge separates the urinal opening into two areas, one of which is closed off and includes a valve only permitting flow of liquid into the urinal. U.S. Pat. No. 5,893,176 discloses a funnel with an inner sealing ring adapted to surround and sealingly engage the exterior of the labia minora and an outer sealing ring adapted to surround and sealingly engage the exterior of the labia majora. U.S. Pat. No. 5,956,782 discloses a urinal device for use by women while seated in a chair or wheelchair. The urinal device has a triangular (i.e., pie-slice) shaped shallow pan with a swollen, bulbous cross-section rim and a perforated splash plate. A handle is affixed to the pan opposite the narrow end of the pan so that the user may insert the pan beneath her body. A drainage hose leads from an outlet to a container. U.S. Pat. No. 6,041,448 discloses an evacuator with a triangular shape. U.S. Pat. No. 6,723,078 discloses a urinal kit with a pair of specifically adapted male and female collector funnels. The male collector funnel forms a cylindrical flexible membrane that has a rollable sidewall and is in fluid communication with a drain tube for connection with a collection conduit. The female collector funnel forms a rigid sidewall terminating at the upper end in an upper sealing ridge forming a contoured upper edge specifically adapted to seal against the user's body when pressed against the user's groin, and is in fluid communication with a drain tube for connection with a collection conduit. U.S. Pat. No. 6,941,587 discloses a urinal with a generally triangular shape for stability. A handle helps users properly position the urinal in a manner that forms a good seal against the relevant portion of the female anatomy.

While the prior art urinals may be effective for their intended purpose, they have shortcomings. Specifically, the prior art devices do not have a backflow prevention device, such as a check valve. Urine and odors may travel unimpeded in both directions through the container and inlet. Such devices are particularly vulnerable to spillage and emitting foul odors. Additionally, the prior art devices are not optimally shaped to sealingly engage a user's groin. The prior art devices are also not optimally configured to capture and redirect a stream of urine discharged from a female urethra into a container without appreciable backsplash. Furthermore, the prior art devices are not adapted to connect to a Foley collection bag, which are widely available and commonly used for collection of urine discharged from Foley catheters. Instead the prior art devices include integral containers or connections to a proprietary collection containers.

The invention is directed to overcoming one or more of the problems and solving one or more of the needs as set forth above.

### SUMMARY OF THE INVENTION

To solve one or more of the problems set forth above, in an exemplary implementation of the basic apparatus of the invention, an external voiding apparatus includes a triangular cup, an outlet at a lower vertex of the cup, a throat extending outwardly from the outlet of the cup, a check valve assembly, and a valve seat. The cup acts as a receiver. The valve assembly is disposed between the throat and the valve seat. A tube and a fluid coupling assembly connect (i.e., fluidly couple) the valve seat to the tube. The fluid coupling assembly includes a funnel coupling from the throat to a tube. A fluid collection container (e.g. a Foley bag) is preferably fluidly coupled to the tube. In addition, in its more complete system embodiments, the invention includes means for storing and implementing the invention that allow it to be easily and conveniently used in both home and institutional settings.

The cup has an open top or side with gradually sloping side walls that rise to and converge on the outlet located adjacent the lower vertex of the cup. The cup has a length between 4 inches and 8 inches, a width between 2 inches and 6 inches, and a depth between 1 inches and 6 inches. Optionally, the cup may include a phosphorescent polymer to provide a glow in the dark effect. Optionally, a handle forms part of the cup for ease of handling, but can also be detachable. A removable cover may be provided to temporarily cover the cup. Finally, a support member suitable for holding the aforesaid apparatus in convenient reach of the user and/or associated care givers completes the system of the invention. This support member is preferably implemented by the provision of a cup support which can be attached to a bedside stand or table (such as a patient hospital table) so as to form the support member. In its preferred embodiments, the system also includes a basket or other similar apparatus which can also be attached to such a bedside stand or table and serves to hold items (such as those useful for the cleaning of the cup and other elements) within ready reach of the user and/or caregiver so as to assist in the implementation of the invention.

A compressible resilient gasket is provided on the top edge of the cup. The gasket is adapted to conform to the contour of a user's groin when pressure is applied. The gasket, which is thicker than the wall thickness of the cup, defines a rim that overhangs the top edge of the cup. The compressible resilient gasket is ideally comprised of an arcuate (e.g., a shallow C-shaped top edge) shaped to conform to the contour of a user.

Likewise, the compressible resilient gasket on the top edge of the cup may also have an arcuate “S”-shaped configuration, shaped to conform to the contour of a user. It may be formed using a closed cell foam as well, but this is not presently considered an optimal design as sanitation problems have arisen from the use of such materials for similar purposes in therapeutic settings.

A mechanical connection couples the valve seat to the tube. The mechanical connection may comprise threads, tabs, and snap fit details.

The check valve assembly includes a valve body with at least one aperture covered by a resilient pliable membrane. The resilient pliable membrane is configured to deflect when urged by discharged liquid. Deflection allows the discharged liquid to travel through the at least one aperture. The membrane may be 5 to 100 mil thick silicone. In another embodiment, the check valve assembly is a duckbill valve.

### BRIEF DESCRIPTION OF THE DRAWINGS

The foregoing and other aspects, objects, features and advantages of the invention will become better understood with reference to the following description, appended claims, and accompanying drawings, where:

FIG. 1 provides an exploded plan view of an exemplary external voiding apparatus according to principles of the invention; and

FIG. 1A provides a section view of section A-A showing a profile of an exemplary “C” shaped gasket for an external voiding apparatus according to principles of the invention; and

FIG. 1B provides a section view of section A-A showing another profile of an “S” exemplary gasket for an external voiding apparatus according to principles of the invention; and

FIG. 2 provides a first perspective view of an exemplary valve assembly for an external voiding apparatus according to principles of the invention; and

FIG. 3 provides a second perspective view of an exemplary valve assembly for an external voiding apparatus according to principles of the invention; and

FIG. 4 provides a first exploded perspective view of an exemplary valve assembly for an external voiding apparatus according to principles of the invention; and

FIG. 5 provides a second exploded perspective view of an exemplary valve assembly for an external voiding apparatus according to principles of the invention; and

FIG. 6 provides a first side view of an exemplary assembled external voiding apparatus with a handle, according to principles of the invention; and

FIG. 7 provides a first exploded perspective view of an exemplary external voiding apparatus according to principles of the invention; and

FIG. 8 provides a second exploded perspective view of an exemplary external voiding apparatus according to principles of the invention; and

FIG. 9 provides a view of female genitalia, including a urethral opening; and

FIG. 10 provides a view illustrating use of an exemplary assembled external voiding apparatus placed over female genitalia according to principles of the invention; and

FIG. 11 illustrates a Foley bag alongside a bed to which the tube of the exemplary external voiding apparatus may connect; and

FIG. 12 provides a perspective view of a support member for holding and supporting the external voiding apparatus and associated items; and

FIG. 13 provides a perspective view of an exemplary detachable handle according to principles of the invention.

Those skilled in the art will appreciate that the figures are not intended to be drawn to any particular scale; nor are the figures intended to illustrate every embodiment of the invention. The invention is not limited to the exemplary embodiments depicted in the figures or the type of valve assembly, handle configuration, shapes, relative sizes, ornamental aspects or proportions shown in the figures.

### DETAILED DESCRIPTION

Referring to the Figures, in which like parts are indicated with the same reference numerals, various views of an exemplary external voiding apparatus 100 according to principles of the invention are shown. The exemplary apparatus includes a generally triangular cup 110, a cup outlet 115, a throat 120, attachment features 130, a throat outlet 125, a valve assembly 135, a valve seat 155, a reservoir 160, a coupling 165 from the reservoir 160 to a narrower diameter tube 170.

The cup 110 receives urine discharged from a urethral opening and guides the urine towards the cup outlet 115. The cup 110 serves as a dam. The cup 110 includes an open top and gradually sloped walls that converge at the outlet 115 at a vertex. The shape of the cup acts as a funnel, capturing and redirecting a stream of discharged urine towards the outlet 115 without excessive splash-back, and is suitable for use with males as well as females (though the principle need for the invention is anticipated to be with females).

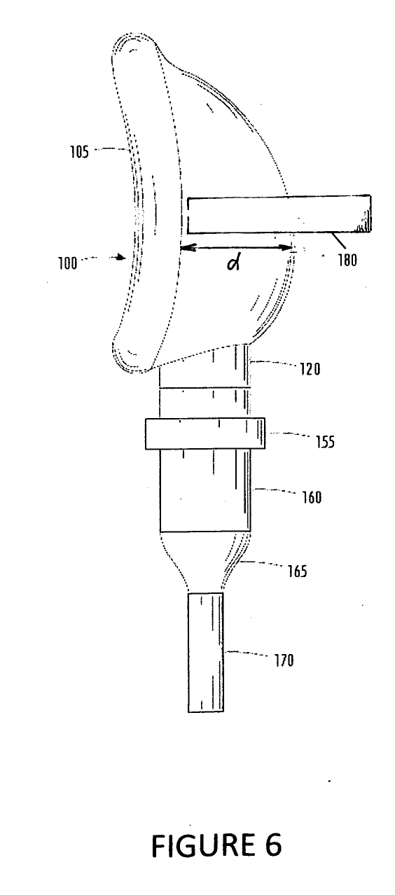

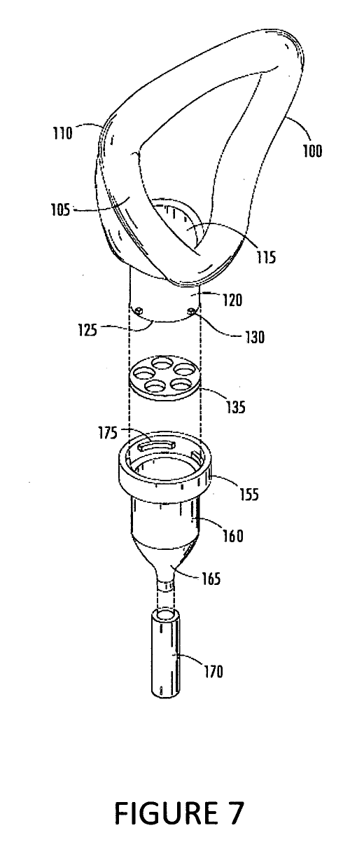

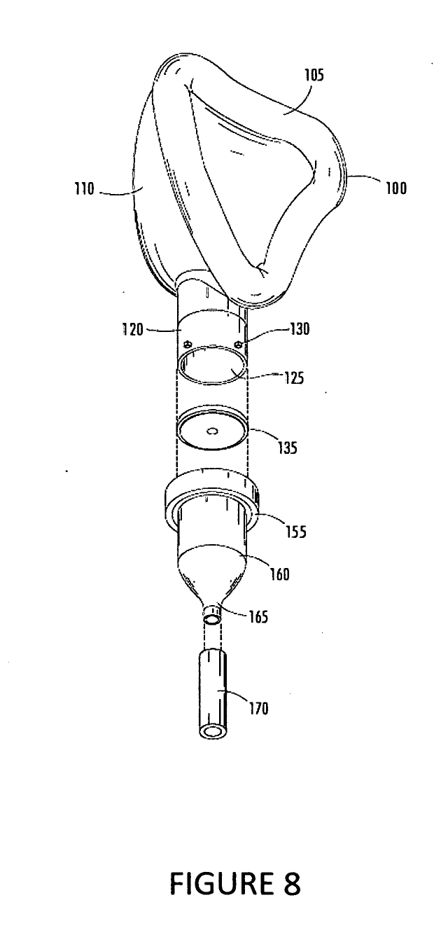

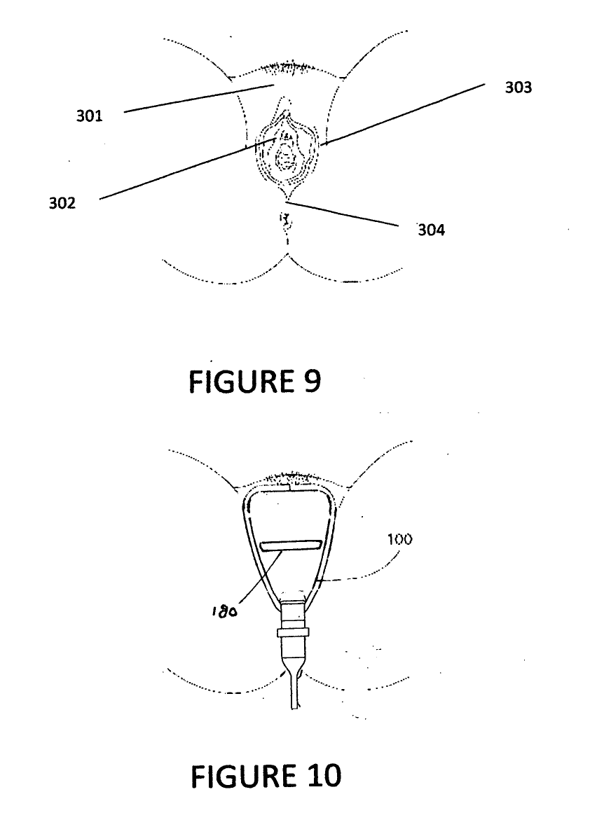

The cup 110 may come in various sizes to accommodate a range of male and female users. In a preferred embodiment the cup 110 is sized to cover an adult female's vulva. In an exemplary embodiment, the length, l, is between 4 inches and 8 inches, preferably about 6 inches and is sized to: (1) stretch from and snugly fit along the arch of the pubic bone or mons pubis 301 at its upper end above the vulva (female genitalia 302); stretch along and snugly fit beside both sides of the female genitalia 302 in the inguinal folds 303 between the legs and female genitalia 302; and (3) reach its lower apex adjacent outlet 115 where it fits below female genitalia 302 at the perineum 304. In an exemplary embodiment, the width, w, is between 2 inches and 6 inches, preferably about 4 inches. In an exemplary embodiment, the depth, d, as shown in FIG. 6, is between 1 inches and 6 inches, preferably about 3 to 4 inches.

The cup 110 is comprised of a substantially rigid material suitable for contact with human flesh and urine. Although any of a variety of metals, plastics and composites may be used, a plastic cup 110 is preferred. In a particular exemplary implementation, the cup 110 is primarily comprised of a rigid plastic or polymeric material, such as polyvinyl chloride (PVC), polyethylene, polypropylene, polystyrene, acrylics, cellulosics, acrylonitrile-butadiene-styrene terpolymers, urethanes, thermo-plastic resins, thermo-plastic elastomers (TPE), acetal resins, polyamides, polycarbonates and polyesters. While many other materials may be used alone or in combination with the aforementioned materials and/or other materials, without departing from the scope of the present invention, preferably the material is relatively inexpensive, easy to use in manufacturing operations and results in an aesthetically acceptable, durable product. The material may further include additives to provide desired properties such as desired colors, structural characteristics, glow-in-the dark properties and thermal reactivity (e.g., color changes according to heat).

By way of example and not limitation, a phosphorescent polymer additive, such as aluminate based phosphors, may be added to some or all parts of the cup 110 to provide a glow in the dark effect (i.e., to adsorb light energy and continue to release that energy as visible light after the energy source is removed). Advantageously, such an embodiment provides a cup 110 that is easy to locate in darkened conditions, making the cup 110 easy to locate even at nighttime.

The cup 110 and its components may be produced using any suitable manufacturing techniques known in the art for the chosen material, such as (for example) injection, compression, structural foam, blow, or transfer molding; polyurethane foam processing techniques; vacuum forming; casting and milling. Preferably, the manufacturing technique is'suitable for mass production at relatively low cost per unit, and results in an aesthetically acceptable product with a consistent acceptable quality.

A compressible resilient gasket 105 covers the top edge of the cup 110. The compressibility of the gasket 105 allows the gasket 105 to conform to the contour of the user's groin when pressure is applied. When pressure is released, the resilient gasket returns to its original shape. The gasket 105 may be attached to the cup via a formed slot and bead arrangement, and is removable and washable.

The height, h, and resiliency of the gasket 105 allow sufficient deformation to accommodate a wide range of body contours. Illustratively, the height, h, may be between ¼ inch and 2 inches, preferably about ½ inch.

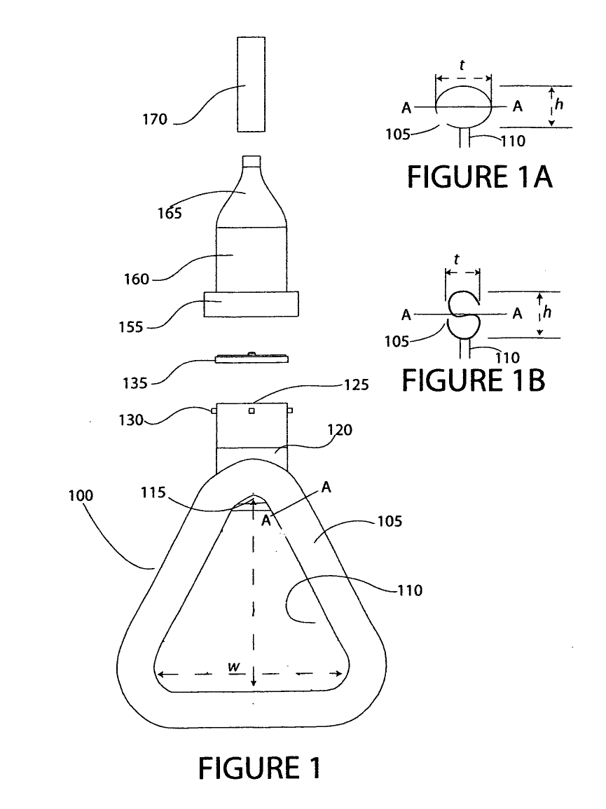

As shown in FIGS. 1A and 1B, the gasket 105 is wider than the sidewall of the cup 110 (i.e., t>wall thickness). Therefore, the gasket provides a rim that prevents liquid traveling up the sidewall of the cup 110 from being discharged past the edge of the cup 110. Illustratively, the thickness, t, may be between ¼ inch and 1 inch, preferably about ½ inch.

The gasket may have various shapes. An ellipsoid C-shaped cross-section is shown in FIG. 1A. An S-shaped cross-section is shown in FIG. 1B. D-shaped, E-shaped, H-shaped, O-shaped and U-shaped gasket profiles are also possible. These and other shapes may be used within the spirit and scope of the invention.

In the preferred embodiment, the gasket 105, and/or the cup 110 may have a contour that is curved to conform to the anatomy of a user. Such a curved contour is illustrated in the drawing figures. The curvature follows the contours of the mons pubis 301 and inguinal folds 303 as described above. The curvature, as seen from a side view, generally resembles a shallow C or a parenthesis, with the fore and aft portions protruding from the cup further than the intermediate portions. It may also be bowed slightly inwardly or outwardly at the sides to better fit into inguinal folds 303 or at its upper end to better fit along the pubic arch 301.

The gasket 105 may be formed from any elastomeric material suitable for constructing a soft, compressible, resilient gasket structure that will contact human tissue and urine. The gasket 105 and its components may be produced using any suitable manufacturing techniques known in the art for the chosen material, such as (for example) extrusion, casting, polyurethane foam processing techniques, casting and milling. Preferably, the manufacturing technique is suitable for mass production at relatively low cost per unit, and results in an aesthetically acceptable product with a consistent acceptable quality.

In use, the gasket creates a seal (e.g., watertight or semi-watertight). The gasket also provides a comfortable surface against the user.

Urine received in the cup 110 is discharged through an outlet assembly. The outlet assembly includes a cup outlet 115. The cup outlet 115 is an aperture in the cup 110, through which liquid may freely flow. A throat 120 is connected to the cup 110 at the cup outlet 115. Liquid flowing through the cup outlet 115, flows into the throat 120. The throat includes an outlet 125 and throat attachment features 130. The features 130 enable interlocking fluid-tight engagement with a mating receptacle that is fluidly coupled to a hose. The features 130 may comprise threads, tabs, snap fit details, or other structures suitable for releasable, fluid tight engagement. Such features may, for example, comprise a quarter turn male thread used to attach a mating receptacle having female quarter turn threads.

A check valve assembly 135 regulates the fluid flow. The check valve assembly 135 is disposed between the throat outlet 125 and the valve seat 125. The valve assembly 135 allows fluid to flow from the outlet 125 through various coupling components and into the tube 170. The valve assembly 135 restricts or prevents flow in the opposite direction. Thus, the valve assembly 135 helps retain discharged liquids and associated odors, thereby preventing or limiting spillage and seepage.

As shown in the drawing figures, an exemplary valve assembly 135 is a flap style valve that includes a thin pliable resilient membrane 150 covering one or more apertures 145 in a valve body 140. Liquid flows through the one or more apertures 145 and impacts the membrane 150, causing the membrane to deflect. The deflection allows the liquid to flow past the membrane and continue its travel. A fastener, such as a screw 152, secures the membrane 150 at one point to the valve body 140.

The membrane 150 may be formed from any elastomeric material suitable for constructing a thin, resilient pliable sheet that will contact human urine. In an exemplary embodiment, a thin silicone sheet is used. However, the invention is not limited to silicone. Other thin pliable resilient materials may be utilized without departing from the scope of the invention. The thickness of the membrane is not particularly important, so long as it retains resiliency, provides a seal and yields under the pressure of discharged urine. Thickness will also depend on the modulus of the material, with thickness decreasing as modulus increases. Modulus will depend in part upon the composition and manufacturing and environmental conditions. By way of example and not limitation, a 5 to 100 mil thick silicone rubber membrane may be used.

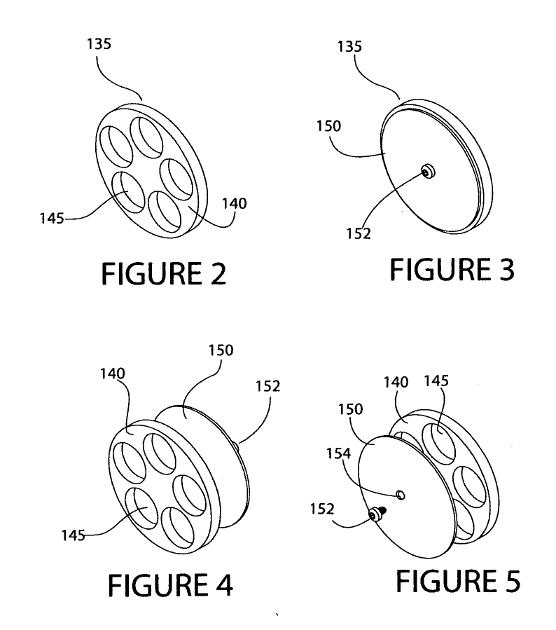

The invention is not, however, limited to the valve assembly shown in the drawing figures. The number and arrangement of apertures may vary. The shape and features of the valve body 140 and membrane 150 may also vary. Other types of check valve assemblies may be used in lieu of or in addition to the valve assembly shown in FIGS. 2 through 5. By way of example and not limitation, a duck bill valve may be utilized to regulate the flow.

A duckbill valve may be manufactured from rubber or synthetic elastomer, and shaped like the beak of a duck. One end of the valve is stretched over the outlet of a supply line, such as throat 120, conforming itself to the shape of the throat. The other end, the duckbill, retains its natural flattened shape. This other end would be contained in reservoir 160. When a fluid is discharged through the valve, the flattened end opens to permit the pressurized fluid to pass. When pressure is removed, the duckbill end returns to its flattened shape, preventing backflow.

The valve assembly 135 sits in the valve seat 155, abutting the reservoir 160. The valve seat 155 also contains threads or other features 175 for locking engagement with the features 130 on the throat 120 of the cup 110. During assembly, the valve assembly 135 is seated and the throat is positioned in the valve seat 155 with the features 130, 175 aligned for engagement. Engagement may be achieved by twisting and or snap-fit action. When engaged, the edge of the throat 120 is secured against the valve assembly 135 and the valve assembly is sandwiched between the throat 120 and the reservoir 160. One or more o-rings or gaskets may be used to ensure a fluid-tight seal. An exemplary assembled apparatus is shown in FIGS. 6 and 10.

The valve seat 155 resides upstream of the reservoir 160 and may be integrally formed with the reservoir 160. The reservoir 160 accumulates and temporarily stores liquid discharged through the valve assembly 135. The reservoir 160 provides a temporary collection compartment until the collected liquid is completely discharged through the tube 170 into a collecting bag or other container. The funnel-like coupling 165 leading to the narrower diameter tube 170 is susceptible to backing up in the presence of a high volumetric flow rate. The reservoir 160 provides a volume to store the backed-up fluid while drainage from the relatively large diameter reservoir 160 to the small diameter tube 170 takes place. The reservoir 160 may be sized to hold an entire volume of urine discharged from a user or hold only a portion of the urine while drainage through the tube 170 takes place.

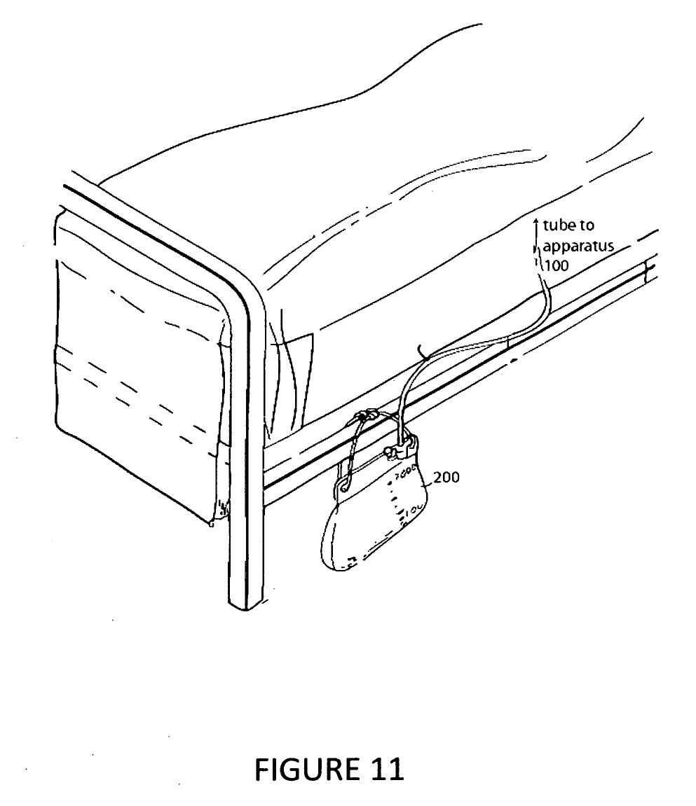

A coupling 165 extends from the reservoir 160 to a narrower diameter tube 170. The coupling 165 fluidly connects the throat to the tube 170, funneling liquid into the tube 170. While only a shortened segment of the tube is shown in the Figures, persons skilled in the art will appreciate that the tube 170 will have a length and be equipped with a coupling for attachment to a urine collection device 200, such as a Foley bag, as conceptually illustrated in FIG. 11. The tube 170 may connect directly to the bag 200 or to a tube connected to the bag 200. Any suitable leak-free coupling may be utilized, including, but not limited to, Luer-Lok® or Luer-Slip® (trademark of Becton, Dickinson and Company Corporation, New Jersey) style fittings, or the like.

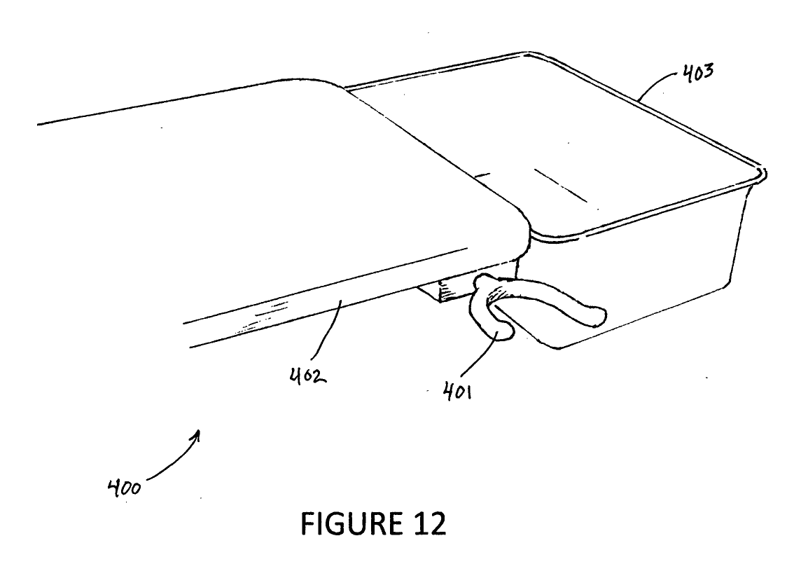

A support member 400 suitable for holding the aforesaid apparatus in convenient reach of the user and/or associated care givers completes the system of the invention. This support member 400 is preferably implemented by the provision of a cup support 401 which can be attached to a bedside stand or table 402 (such as a patient hospital table) so as to form the support member. The cup support can, as illustrated in FIG. 12, advantageously take the form of a horizontal “U” shaped member having a width between its arms less than “W” such than the external voiding apparatus 100 can easily be placed between said arms and supported. In its preferred embodiments, the system also includes a basket 403 or other similar apparatus which can also be attached to such a bedside stand or table and serves to hold items (such as those useful for the cleaning of the cup and other elements) within ready reach of the user and/or caregiver so as to assist in the implementation of the invention.

In FIG. 6 an embodiment with an optional handle 180 is shown. The handle 180 facilitates gripping and holding the cup 110. The handle 180 may also be used to hang the cup 110 from a bed rail, night table handle, or other support, such as cup support 401. The handle may be ergonomically configured and fitted or overmolded with a comfortable hand grip. Preferably, as illustrated in the drawing figures, the handle 180 should be positioned horizontally as shown in the drawing figures for ease in gripping the handle by the user and/or by someone giving assistance at the bedside. It facilitates the user and/or care giver in pressing the cup 110 firmly into sealing position prior to and during use.

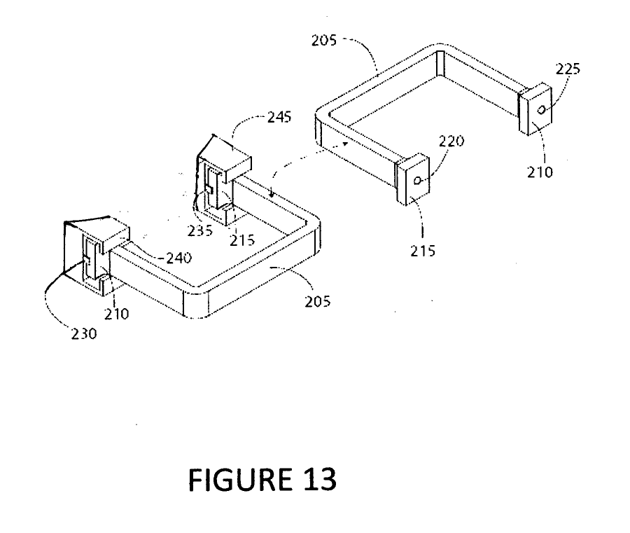

Another embodiment with a removable handle 205 is shown in FIG. 13. In this embodiment the handle 205 is releasably attached to the fore section of the cup 110. The handle includes a pair of tabs 210, 215 configured for releasable engagement in corresponding grooves 240, 245 that are molded as part of or otherwise joined to the cup 110. The tabs also include features (e.g., a slight protuberances 210, 215) configured to fit in complementary (e.g., a slight depression) feature in groove 240, 245, to register for locking engagement. Optional stops 230, 235 are also provided on one side of the groove 240, 245 to prevent sliding past the end of the groove 240, 245.

Although use with female vulva/genitalia 301 is illustrated, persons of skill in the art will appreciate that a male person may use the apparatus by directing the penis towards the outlet 115 of the cup 110. The shape of the cup 110 accommodates various body positions. In each case, the outlet 115 is preferably tilted downward to facilitate the flow of discharged urine under the influence of gravity.

During use, urine flows from the individual into the cup 110. The gasket 105 prevents leakage between the user and the cup 110. The urine is guided by the contour of the cup 110 and the influence of gravity to the outlet of the cup 110, through the throat 120, through the check valve 135, and into the reservoir 160. The check valve 135 opens under the pressure of the urine. From the reservoir, urine drains through the funnel-like coupling 165 into the tube 170. From the tube 170, the urine may travel to a collection container, such as a Foley bag. When urination is complete, the cup 110 may be cleaned and covered using an optional cover. Additionally, while the apparatus is draining into a collection bag, the cup 110 may be hung on cup support 401 as described above or by using optional handle 180, 205.

All mechanical (e.g., threaded) fittings are universal to fit the remaining parts of the apparatus 100 regardless of size. This facilitates maintenance and assembly.

A cover for the open portion of cup 110 may also be provided. The cover may be rigid or flexible. By way of example, a flexible plastic bag-like cover with elastic to attach like a shower cap may be utilized. Alternatively, a molded rigid plastic cover sized to fit over the opening of the cup 110, may be utilized. The cover may be removed, cleaned, discarded and replaced. The cover prevents any spillage of urine residue and facilitates bedside storage and cleanliness until the apparatus is needed.

While an exemplary embodiment of the invention has been described, it should be apparent that modifications and variations thereto are possible, all of which fall within the true spirit and scope of the invention. With respect to the above description then, it is to be realized that the optimum relationships for the components and steps of the invention, including variations in order, form, content, function and manner of operation, are deemed readily apparent and obvious to one skilled in the art, and all equivalent relationships to those illustrated in the drawings and described in the specification are intended to be encompassed by the present invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

What is claimed is:

1. An external voiding apparatus comprising a substantially triangular cup, an outlet at a lower vertex of the cup, a throat extending outwardly from the outlet of the cup, a check valve assembly, a valve seat, said valve assembly being disposed between said throat and said valve seat, a tube and a fluid coupling assembly fluidly coupling the valve seat to the tube.

2. An external voiding apparatus according to claim 1, said cup including an open top and gradually sloped walls that converge at the outlet at a vertex.

3. An external voiding apparatus according to claim 2, said cup having a length between 4 inches and 8 inches, a width between 2 inches and 6 inches, and a depth between 1 inches and 6 inches.

4. An external voiding apparatus according to claim 2, said cup including a phosphorescent polymer in an amount effective to provide a glow in the dark effect.

5. An external voiding apparatus according to claim 2, said cup having a top edge, and said apparatus further comprising a compressible resilient gasket on the top edge of the cup.

6. An external voiding apparatus according to claim 2, said cup having a top edge, and said apparatus further comprising a compressible resilient gasket on the top edge of the cup, said gasket being adapted to conform to the contour of a user's groin when pressure is applied.

7. An external voiding apparatus according to claim 2, said cup having a top edge, and said apparatus further comprising a compressible resilient gasket on the top edge of the cup, said gasket defining a rim that overhangs the top edge of the cup.

8. An external voiding apparatus according to claim 2, said cup having a top edge, and said apparatus further comprising a compressible resilient gasket on the top edge of the cup, said compressible resilient gasket having a thickness that exceeds a thickness of the top edge of the cup, and said gasket defining a rim that overhangs the top edge of the cup.

9. An external voiding apparatus according to claim 2, said cup having an arcuate top edge shaped to conform to the contour of a user.

10. An external voiding apparatus according to claim 2, said cup having a top edge, and said apparatus further comprising a compressible resilient gasket on the top edge of the cup, said compressible resilient gasket having an arcuate configuration shaped to conform to the contour of a user.

11. An external voiding apparatus according to claim 2, said fluid coupling assembly comprising a funnel coupling from the throat to a tube.

12. An external voiding apparatus according to claim 2, further comprising a mechanical connection configured to fluidly coupling the valve seat to the throat, said mechanical connection comprising a feature from the group consisting of threads, tabs, and snap fit details.

13. An external voiding apparatus according to claim 2, said check valve assembly comprising a valve body with at least one aperture covered by a resilient pliable membrane, said resilient pliable membrane being configured to deflect when urged by discharged liquid, thereby allowing the discharged liquid to travel through the at least one aperture.

14. An external voiding apparatus according to claim 13, said membrane comprising 5 to 100 mil thick silicone.

15. An external voiding apparatus according to claim 2, said check valve assembly comprising a duckbill valve.

16. An external voiding apparatus according to claim 2, further comprising a fluid collection container fluidly coupled to said tube.

17. An external voiding apparatus according to claim 16, said fluid collection container comprising a bag.

18. An external voiding apparatus according to claim 2, further comprising a handle attached to the cup.

19. An external voiding apparatus according to claim 2, further comprising a support member suitable for holding the cup in spaced relationship to a floor.

20. An external voiding apparatus according to claim 19, wherein said support member comprises at least one of a cup support which can be attached to a bedside stand, and a basket which can also be attached to a bedside stand.
